(19) United States
(12) Patent Application Publication
(10) Pub. No.: US 2024/0247318 A1
(43) Pub. Date: Jul. 25, 2024

(54) METHODS FOR DETECTING INACTIVATION OF THE HOMOLOGOUS RECOMBINATION PATHWAY (BRCA1/2) IN HUMAN TUMORS

(71) Applicants: Institute Curie, Paris (FR); INSERM (Institut National de la Sante et de la Recherche Medicale), Paris (FR)

(72) Inventors: Marc-Henri Stern, Paris (FR); Elodie Manie, Paris (FR); Tatiana Popova, La Varenne Saint Hilaire (FR)

(73) Assignees: Institute Curie, Paris (FR); INSERM (Institut National de la Sante et de la Recherche Medicale), Paris (FR)

(21) Appl. No.: 18/442,056

(22) Filed: Feb. 14, 2024

Related U.S. Application Data
(63) Continuation of application No. 17/381,272, filed on Jul. 21, 2021.
Application No. 17/381,272 is a continuation of application No. 14/402,254, filed on Mar. 25, 2015, now Pat. No. 11,091,808.

(30) Foreign Application Priority Data
Jun. 7, 2012 (EP) 12305648.3

(51) Int. Cl. C12Q 1/6886; A61K 31/131; A61K 31/166; A61K 31/282; A61K 31/407; A61K 31/454; A61K 31/502; A61K 31/5025; A61K 31/55; A61K 33/243; C12Q 1/6827

(52) U.S. Cl. CPC C12Q 1/6886; A61K 31/131; A61K 31/166; A61K 31/282; A61K 31/407; A61K 31/454; A61K 31/502; A61K 31/5025; A61K 31/55; A61K 33/243; C12Q 1/6827; C12Q 2600/112; C12Q 2600/154; C12Q 2600/156

(57) ABSTRACT

The invention relates to methods for detecting inactivation of the DNA Homologous Recombination pathway in a patient, and in particular for detecting BRCA1 inactivation.

### CROSS REFERENCE TO RELATED APPLICATIONS

This application is a continuation of U.S. patent application Ser. No. 17/381,272, filed Jul. 21, 2021, which is a continuation of U.S. patent application Ser. No. 14/402,254, filed Nov. 19, 2014, now U.S. Pat. No. 11,091,808, which is a U.S. National Phase application under 35 U.S.C. § 371 of International Application No. PCT/EP2013/061707, filed Jun. 6, 2013, which claims the benefit of and priority to European Patent Application No. 12305648.3, filed Jun. 7, 2012, the contents of which are incorporated herein by reference in their entirety.

### FIELD OF THE INVENTION

The invention relates to methods for detecting a predisposition to develop cancer and methods for treating cancer.

### BACKGROUND OF THE INVENTION

Cancer is a class of diseases in which a group of cells display the traits of uncontrolled growth (growth and division beyond the normal limits), invasion (intrusion on and destruction of adjacent tissues), and sometimes metastasis (spread to other locations in the body via lymph or blood). Cancers can be classified according to the organ, tissue and cell-type from which the cancerous cells originate: lung, colon, liver, skin etc.

Cancer represents one of the leading causes of death in the world. Successful treatment relies on the diagnosis of the disease at very early stages and on the choice of adapted therapies. A plurality of risk factors (lifestyle related, genetic etc.) has been identified for certain types of cancers.

Breast cancer (malignant breast neoplasm) is a type of cancer originating from breast tissue, most commonly from the inner lining of milk ducts or the lobules that supply the ducts with milk. Cancers thought to be originating from ducts are known as ductal carcinomas; those thought to be originating from lobules are known as lobular carcinomas.

Basal-like breast carcinomas (BLCs) are generally described as high grade ductal carcinomas, having so-called triple negative (TNBC) phenotype (absence of estrogen receptor [ER], progesterone receptor [PR] and HER2/ERBB2 overexpression) and characterized by the markers expressed by the normal basal/myoepithelial cells of the mammary gland (such as cytokeratins 5/6, 14, 17 and EGFR (for review,^{1,2}).

Breast cancer susceptibility gene BRCA1 has a particular connection to the basal-like phenotype: firstly, BLCs represent the majority of breast carcinomas developing in BRCA1 mutation carriers, while being less than 20% in a sporadic context;³ secondly, high level of genomic instability observed in BLCs4-6 goes in line with BRCA1 involvement in double strand break (DSB) signaling and repair by homologous recombination (HR) (for review,^{7,8}). However, since HR deficiency (so called BRCAness) has been proposed as a general feature of BLCs9, such hallmarks as BRCA1 inactivation, high level of genomic instability, and potentially therapeutic response to the treatment exploiting HR deficiency, were found relevant to merely half of BLCs (or TNBCs).¹⁰⁻¹⁵

Considering its importance in diagnosis and therapeutic stratification, numerous studies attempted to define clinically relevant surrogate markers of BRCAness (for review,¹⁶). Genomic markers of BRCAness were mainly searched by comparing array-CGH profiles of BRCA1 mutated versus unselected hereditary or sporadic breast tumors.¹⁷⁻²¹ Studies comparing BLCs with or without BRCA1 inactivation either found no difference^{11,22,23} or identified 3q gain as associated with BRCA1 inactivation¹². Array-CGH classifier trained on BRCA1 mutated tumors within unselected group of tumors²⁴ showed approximately 80% sensitivity in the TNBC subgroups in two independent studies.^{25,26}

Thus, there is still an unfulfilled need in the art for methods for genomic markers predicting actual BRCA1 inactivation within the group of basal-like breast carcinomas and other cancers.

Recently, Birkbak et al. have described a method for predicting defective DNA repair and response to DNA-damaging agents⁶⁰. This method, called Telomeric allelic imbalance (TAI) is based on the number of allelic imbalances extending to the telomeric end of the chromosome. The main problem of this approach is that it takes into account only chromosomal breaks that lead to telomeric allelic imbalance. Thus, many chromosomal breaks will not contribute to the score, impairing its robustness. Another caveat is that allelic imbalance or loss of heterozygosity of chromosomes prevents detection of telomeric allelic imbalance.

Another recent technology, published as US2012/0015050 (Abkevich), focuses on the loss of heterozygosity as a possible predictive marker of homologuous repair defects in epithelial ovarian cancer. These authors propose to calculate the Homologous Recombination Deficiency score (or “HDR score”), which takes into account the number of regions in which there is a loss of heterozygosity (i.e. only one of the alleles is present). This score does not take into account chromosomal breakpoints or rearrangements which result in allelic imbalance.

### SUMMARY OF THE INVENTION

The inventors have discovered that large-scale chromosome breaks are strongly predictive of Homologous Recombination (HR) deficiency, whichever the mechanism of inactivation.

Hence, in one aspect, the invention relates to a method for predicting deficiency in the DNA homologous recombination (HR) pathway in a patient suffering from cancer, comprising the step of quantifying the number of rearrangements in the genomic DNA of a tumor sample obtained from said patient, wherein the number of rearrangements corresponds to the number, per genome, of breakpoints resulting in segments of at least 3 megabases, preferably at least 4 megabases, even more preferably at least 5, 6, 7, 8 9, 10, 11 12, 13, 14, 15, 16, 17, 18, 19 or 20 megabases.

The invention also relates to a method for predicting the efficacy of a treatment in a patient suffering from cancer, wherein said treatment comprises a PARP inhibitor and/or an alkylating agent, and wherein said method comprises the step consisting of predicting deficiency on the HR pathway as described above.

The invention also relates to a PARP inhibitor and/or an alkylating agent for use in a method for treating cancer in a patient wherein said cancer is linked to deficiency in the HR pathway.

The invention also relates to a method for treating cancer in a patient, comprising administering a therapeutically effective amount of a PARP inhibitor and/or an alkylating agent, wherein said patient has been classified as having a deficiency in the HR pathway as described above.

### DETAILED DESCRIPTION OF THE INVENTION

### Methods for Predicting Deficiency in the DNA Homologous Recombination Pathway

In one aspect, the invention relates to a method for predicting deficiency in the DNA homologous recombination (HR) pathway in a patient suffering from cancer, comprising the step of quantifying the number of rearrangements in the genomic DNA of a tumor sample obtained from said patient, wherein the number of rearrangements corresponds to the number, per genome, of breakpoints resulting in segments of at least 3 megabases, preferably at least 4 megabases, even more preferably at least 5, 6, 7, 8 9, 10, 11 12, 13, 14, 15, 16, 17, 18, 19 or 20 megabases.

Typically, the method of the invention comprises the step of comparing the number of rearrangements per genome to a threshold, wherein a number of rearrangements per genome superior to said threshold is indicative of HR deficiency.

As used herein, the term “patient” denotes a mammal, such as a rodent, a feline, a canine, a bovine, an equine, a sheep, a porcine and a primate. Preferably, a patient according to the invention is a human.

The inventors have observed that tumors from patients suffering from BRCA1 mutations or other deficiencies in the DNA Homologous Recombination pathway are characterized by a genome that contains greater number of breakpoints than control samples or tumors from patients suffering from cancers which do not involve the HR pathway.

More specifically, the inventors have demonstrated that the relevant breakpoints are those which result in genomic DNA segments of at least 10 megabases. According to the invention, the breakpoints which result in smaller segments are not taken into account.

Without wishing to be bound by theory, the inventors believe that, by eliminating the breakpoints resulting in segments of less than 3 megabases, preferably of less than 4 megabases, even more preferably of less than 5, 6, 7, 8, 9, 10, 11 12, 13, 14, 15, 16, 17, 18, 19 or 20 megabases the resulting number of breakpoints (or large-scale transitions) is a more accurate measurement of the genomic instability related to homologous recombination deficiency. Other breakpoints with local concentration are not correlated with the homologous recombination status.

As used herein, the expression “DNA homologous recombination (HR) pathway” has its general meaning in the art. It refers to the pathway through which Double Stranded DNA breaks (DSB) are repaired by a mechanism called Homologous Recombination.

Inside mammalian cells, DNA is continuously exposed to damage arising from exogenous sources such as ionizing radiation or endogenous sources such as byproducts of cell replication. All organisms have evolved different strategies to cope with these lesions. One of the most deleterious forms of DNA damage is DSB. In mammalian cells, there are two major pathways to repair DSB: Homologous recombination (HR) and Non Homologous End Joining (NHEJ). HR is the most accurate mechanism to repair DSB because it uses an intact copy of the DNA from the sister chromatid or the homologous chromosome as a matrix to repair the break.

BRCA1, BRCA2, PALB2/FANCN, BRIP1/FANCJ, BARD1, RAD51 and RAD51 paralogs (RAD51B, RAD51C, RAD51D, XRCC2, XRCC3) are proteins that are important for the repair of double-strand DNA breaks by this error-free HR pathway. When the gene for either protein is mutated, or when one of the genes is under-expressed, the change can lead to errors in DNA repair that can eventually cause cancer. Although not yet found recurrently mutated in human tumors, other actors of the HR pathway may potentially be deregulated in cancers, such as FANCA, FANCB, FANCC, FANCD2, FANCE, FANCG, FANCI, FANCL, FANCM, FAN1, SLX4/FANCP or ERCC1.

Thus, the expression “deficiency in the HR pathway”, as used herein, refers to a condition in which one or several of the proteins involved in the HR pathway for repairing DNA is deficient or inactivated.

It encompasses, but is not limited to, inactivation of at least one of the following genes: BRCA1, BRCA2, PALP2/FANCN, BRIP1/FANCJ, BARD1, RAD51, RAD51 paralogs (RAD51B, RAD51C, RAD51D, XRCC2, XRCC3), FANCA, FANCB, FANCC, FANCD2, FANCE, FANCG, FANCI, FANCL, FANCM, FAN1, SLX4/FANCP and ERCC1.

A used herein the expressions “deficiency in the HR pathway” or “tumor deficiency in the HR pathway” are used interchangeably. Indeed, it refers to the genetic status of the tumor cells. However, in the case of germline mutations, said mutations can be found throughout the entire genome of the patient.

As used herein the term “inactivation”, when referring to a gene, can mean any type of deficiency of said gene. It encompasses germline mutations in the coding sequence, somatic mutations in the coding sequence, mutations in the promoter and methylation of the promoter.

In one embodiment of the invention, the deficiency in the HR pathway is a BRCA1 mutation. Several BRCA1 mutations have already been described in the art and are known to be associated with certain types of cancer, such as breast and ovary cancers⁵⁵.

On another embodiment of the invention, the deficiency in the HR pathway is a BRCA2 mutation⁵⁶.

In yet another embodiment of the invention, the deficiency in the HR pathway is hypermethylation of the BRCA1 promoter⁵⁷.

As used herein, the term “cancer” has its general meaning in the art. It refers to the pathological condition in mammals that is characterized by unregulated cell growth. Examples of cancer include, but are not limited to solid tumors or a carcinoma. Preferably, the solid tumor is selected from breast cancer, colon cancer, lung cancer, prostate cancer, renal cancer, metastatic or invasive malignant melanoma, brain tumor, bladder cancer, head and neck cancer and liver cancer. Carcinoma includes bladder, breast, colon, kidney, liver, lung, ovary, pancreas, stomach, cervix, thyroid or skin carcinoma, including squamous cell carcinoma. However, the present invention also contemplates hematopoietic tumors such as leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, Burkitt's lymphoma, acute and chronic myelogenous leukemias and promyelocytic leukemia.

In one embodiment, said cancer is selected from the group consisting of breast cancer, ovary cancer, pancreas cancer, head and neck cancer and melanoma.

In a preferred embodiment, said cancer is selected from the group consisting of breast cancer, ovary cancer, cervix cancer, pancreas cancer and lung cancer.

In a more preferred embodiment, said cancer is a breast cancer.

The tumor sample suitable for carrying out the method of the invention is typically a biopsy obtained from the diseased tissue or organ of the patient suffering from cancer.

### Quantification of the Number of Rearrangements

The step of quantifying the number of rearrangements per genome in the genomic DNA of the tumor sample can be performed by any suitable method in the art.

As mentioned above, the inventors have demonstrated that the relevant breakpoints are those which result in genomic DNA segments of at least 3 megabases. Indeed, preferred cut-off points comprised between 9 and 11, even more preferably about 10 megabases, have been described in the Examples below, but similar results were obtained with cutoff value between 3 megabases and 20 megabases. According to the invention, the breakpoints which result in segments of less than these cutoff points are not taken into account.

The skilled person can readily select any method for quantifying genomic rearrangements and filter out the breakpoints that result in genomic DNA segments of less than 3, preferably less than 4, 5, 6, 7, 8, 9, 10, 11, 12, 13, 14, 15, 16, 17, 18, 19, or 20 megabases.

In one embodiment, the step of quantifying rearrangements is carried out by sequencing techniques, such as next-generation sequencing using mate paired libraries, or longer reads.⁵⁸

In another embodiment, the step of quantifying rearrangements is performed by quantifying the number of copy number variations per genome. Typically, this can be done by hybridization techniques such as comparative genomic hybridization (CGH) array and Single Nucleotide Polymorphism (SNP) array.

Suitable methods for quantifying rearrangements include, but are not limited to, those described in Le Scouarnec and Gribble, Heredity, 2012, 108, 75-85.

### Evaluation of the Ploidy

In one embodiment, the method of the invention further comprises a step wherein the ploidy of the tumor sample is evaluated.

As used herein, the term “ploidy” has its general meaning in the art. It refers to the mean number of copies of each locus in the genome.

Typically, a healthy cell (and therefore a healthy tissue sample) is diploid, i.e. it contains two copies/two alleles of each locus.

Without wishing to be bound by theory, it is believed that certain types of cancer are characterized by whole genome duplication during cancer progression, resulting in over-diploid (tetraploid or more) tumor cells (Ref 40).

Tumor samples can be split into diploid tumors or near-diploid tumors on the one hand and over-diploid tumors in the other hand.

The inventors have observed that near-diploid tumor genomes were associated in more than 75% of the cases with BRCA1 inactivation (by mutation or by promoter methylation).

Without wishing to be bound by theory, it is believed that diploid or near-diploid tumors are highly predictive of HR-deficient tumors, at least in high grade breast carcinoma.

Typically, a tumor is deemed to be “diploid or near-diploid” if the genome of said tumor carries on average less than 50 chromosomes and/or if had a DNA index close to 1.

Typically, a tumor is considered as “over-diploid” if its genome carries more than or equal to 50 chromosomes and/or has a DNA index higher than 1.2.

As used herein, the term “DNA index” represents the ratio of DNA content of the tumor cell and DNA content of a normal cell.

The skilled person can evaluate the ploidy of a tumor sample according to any standard technique in the art.

Suitable techniques for evaluating ploidy include, but are not limited to:
Measuring the amount of DNA per cell, by example by Fluorescence Activated Cell Sorting.

In this technique, DNA is labeled by incorporation of an intercalating agent such as ethidium bromide or DAPI. The cells are then sorted according to the fluorescence intensity, which is proportional to the amount of DNA in each cell.
karyotyping,

Conventional karyotypes can be obtained by staining the chromosomes (with stains such as Giemsa) and counting the number of chromosomes of each type in a cell.
Virtual karyotyping using arrays such as array-CGH or Single Nucleotide Polymorphism array (SNP array).

The arrays themselves can be genome-wide (probes distributed over the entire genome) or targeted (probes for genomic regions known to be involved in a specific disease) or a combination of both. Further, arrays used for karyotyping may use non-polymorphic probes, polymorphic probes (i.e., SNP-containing), or a combination of both. Non-polymorphic probes can provide only copy number information, while SNP arrays can provide both copy number and loss-of-heterozygosity (LOH) status in one assay. Commercially available oligonucleotide SNP arrays can be solid phase (Affymetrix, Santa Clara, CA, USA) or bead-based (Illumina, SanDiego, CA, USA). Despite the diversity of platforms, ultimately they all use genomic DNA from disrupted cells to recreate a high resolution karyotype in silico. The end product does not yet have a consistent name, and has been called virtual karyotyping, digital karyotyping, molecular allelokaryotyping, and molecular karyotyping. Other terms used to describe the arrays used for karyotyping include SOMA (SNP oligonucleotide microarrays) and CMA (chromosome microarray).
Next Generation Sequencing.

High throughput methods for sequence the genome or the complete coding region are available. Whole genome or exome deep sequencing approaches can generate copy number and allelic imbalance profiles similar to or even more precise than SNP arrays.

According to one embodiment of the invention, the step of evaluating the ploidy of the tumor sample is carried out by a technique selected from the group consisting of FACS, karyotyping, and SNP array.

In one embodiment, both the step of evaluating the ploidy and the step of quantifying the number of large-scale rearrangement are performed by SNP array.

In a preferred embodiment, both the step of evaluating the ploidy and the step of quantifying the number of large-scale rearrangement are performed by SNP array, followed by GAP analysis.

Genome Alteration Print (GAP) is a bioinformatics tool which has been developed by Popova et al. (Genome Biology, 2009, 10:R128) for automatic detection of absolute segmental copy numbers and genotype status in complex cancer genome profiles measured with SNP-array. This method performs well even for poor-quality data, low tumor content and highly rearranged tumor genomes.

### Two-Step Method

In one embodiment of the invention, the method comprises the step of comparing the number of rearrangements per genome to a threshold, wherein a number of rearrangements resulting in segments of at least 3 megabases (preferably at least 4, 5, 6, 7, 8, 9, 10, 11, 12, 13, 14, 15, 16, 17, 18, 19 or 20) superior to said threshold is indicative of HR deficiency.

Typically, the threshold can have different values, depending on the ploidy of the tumor.

Thus, in a preferred embodiment, the method comprises the step comparing the number of rearrangements in the genomic DNA to a threshold,
wherein said threshold has a first value (threshold1) if the tumor is diploid or near-diploid
and wherein said threshold has a second value (threshold2) if the tumor is over-ploid.

Typically, threshold1 (as determined using segments longer than 10 megabases, threshold value being dependent of the chosen segment size) can be 15 Large-Scale Transitions (LST) per genome, preferably 16, even more preferably 17, 18, 19 or 20 LST per genome.

Typically, the value of threshold1 may vary, depending on how the number of rearrangements or LSTs is defined. Hence, in one embodiment of the invention, threshold 1 for diploid or near-diploid tumors is defined as follows:
if the number of LSTs is defined as the number of rearrangements resulting in segments of at least 6 megabases, threshold1 may be 17, 18 or 19;
if the number of LSTs is defined as the number of rearrangements resulting in segments of at least 7 megabases, threshold1 may be 15, 16 or 17;
if the number of LSTs is defined as the number of rearrangements resulting in segments of at least 8 megabases, threshold1 may be 14
if the number of LSTs is defined as the number of rearrangements resulting in segments of at least 9 megabases, threshold1 may be 11, 12, 13 or 14;
if the number of LSTs is defined as the number of rearrangements resulting in segments of at least 10 megabases, threshold1 may be 11.

Typically, threshold2 (as determined using segments longer than 10 megabases, threshold value being dependent of the chosen segment size) can be 20 Large-Scale Transitions (LST) per genome, preferably 21, even more preferably 22, 23, 24 or 25 LST per genome.

Typically, the value of threshold2 may vary, depending on how the number of rearrangements or LSTs is defined. Hence, in one embodiment of the invention, threshold 2 for overploid tumors is defined as follows:
if the number of LSTs is defined as the number of rearrangements resulting in segments of at least 6 megabases, threshold1 may be 32;
if the number of LSTs is defined as the number of rearrangements resulting in segments of at least 7 megabases, threshold1 may be 27, 28 or 29;
if the number of LSTs is defined as the number of rearrangements resulting in segments of at least 8 megabases, threshold1 may be 26;
if the number of LSTs is defined as the number of rearrangements resulting in segments of at least 9 megabases, threshold1 may be 19, 20, 21, 22, 23, 24 or 25;
if the number of LSTs is defined as the number of rearrangements resulting in segments of at least 10 megabases, threshold1 may be 18, 19, 20, 21, 22.

It falls within the ability of the skilled person in the art to determine the optimum thresholds, depending on the size of the LSTs, in order to arrive at optimal specificity and sensitivity according to the tumor type. For example, optimum thresholds for breast carcinoma are 7/17/29, 8/14/26, 9/14/29 or 10/11/22, whereas optimum threshold in ovarian carcinoma is 6/19/32 or 7/17/29 (LST number/threshold1/threshold2).

Indeed, the inventors have found that a 2-step decision rule, wherein the patients are classified according to the ploidy of the tumor, and according to the number of large-scale transitions in the tumor genome, was able to correctly predict HR deficient tumors.

The invention therefore relates to a method comprising the steps of:
determining the ploidy of the tumor;
comparing the number of rearrangements per genome to a threshold, wherein a number of rearrangements resulting in segments of at least 3 megabases superior to said threshold is indicative of HR deficiency.

Advantageously, the method according to the invention is able to predict deficiency in the HR pathway with good specificity (few false positives) and good sensitivity (few false negatives).

### Methods for Predicting the Efficacy of a Treatment and Methods of Treatment

The method described above has several major and direct clinical applications. Firstly, tumor genomic profiling can now be used as criteria for genetic testing and council. This is especially important in absence of familial context of tumor predisposition, a situation found in as much as half of mutation-carrier patients⁵³.

Secondly with the emerging therapeutic perspective exploiting HR defects by targeting complementary pathways (for instance, PARP inhibitors (PARPi)¹³, and alkylating agents, which provoke DNA damage), the question of efficient predictive markers of BRCAness or HR deficiency becomes important¹⁶. The disappointing efficiency of PARPi in unselected BLC/TNBC⁵⁴ supports the necessity to better stratify patients, which could be easily implemented using this SNP-array based marker.

Since it is possible to predict whether a given patient suffers from a cancer which is associated with deficiency in the DNA homologous recombination pathway, it is also possible to select the appropriate therapy for said patient.

Indeed, it is believed that a treatment which causes double strand breaks in the DNA (such as alkylating agents) or a treatment which inhibits the alternative DNA repair pathway (such as PARPi) will be more efficient if the tumor is deficient for the HR pathway.

In addition, the inventors have shown that the number of LSTs is a good predictor or response to treatment with an alkylating agent such as cisplatin (see Example 3).

Therefore, another aspect of the present invention concerns a method for predicting the efficacy of a treatment in a patient suffering from cancer, wherein said treatment comprises a PARPi and/or an alkylating agent, and wherein said method comprises the step consisting of predicting deficiency on the HR pathway as described above.

The invention also relates to a PARPi and/or an alkylating agent for use in a method for treating cancer in patient wherein said cancer is linked to deficiency in the HR pathway.

As used herein the term “PARP inhibitor” has its general meaning in the art. It refers to a compound which is capable of inhibiting the activity of the enzyme polyADP ribose polymerase (PARP), a protein that is important for repairing single-strand breaks (‘nicks’ in the DNA). If such nicks persist unrepaired until DNA is replicated (which must precede cell division), then the replication itself will cause double strand breaks to form. Drugs that inhibit PARP cause multiple double strand breaks to form in this way, and in tumors with BRCA1, BRCA2 or PALB2 mutations these double strand breaks cannot be efficiently repaired, leading to the death of the cells. Typically, the PARP inhibitor according to the invention can be selected from the group consisting of iniparib, olaparib, rocaparib, CEP 9722, MK 4827, BMN-673, and 3-aminobenzamide.

As used herein, the term “alkylating agent” or “alkylating antineoplastic agent” has its general meaning in the art. It refers to compounds which attach an alkyl group to DNA.

Typically, the alkylating agent according to the invention can be selected from platinum complexes such as cisplatin, carboplatin and oxaliplatin, chlormethine, chlorambucil, melphalan, cyclophosphamide, ifosfamide, estramustine, carmustine, lomustine, fotemustine, streptozocin, busulfan, pipobroman, procarbazine, dacarabazine, thiotepa and temozolomide.

The invention also relates to a method for treating cancer in a patient, comprising administering a therapeutically effective amount of a PARP inhibitor and/or an alkylating agent, wherein said patient has been classified as having a deficiency in the HR pathway as described above.

In one aspect, the invention relates to a method for treating cancer in a patient, comprising the steps of:
quantifying the number of rearrangements in the genomic DNA of a tumor sample obtained from said patient, wherein the number of rearrangements corresponds to the number, per genome, of breakpoints resulting in segments of at least 3 megabases, preferably at least 4 megabases, even more preferably at least 5, 6, 7, 8 9, 10, 11 12, 13, 14, 15, 16, 17, 18, 19 or 20 megabases.
comparing said number of rearrangements to a predetermined threshold;
administering a therapeutically effective amount of a PARP inhibitor and/or an alkylating agent, if said patient has a number of rearrangements superior to said threshold.

As explained above, said threshold may differ, depending on whether the tumor is a diploid or near-diploid tumor, or rather an overploid tumor.

Said threshold may also differ, depending on the minimum size of the segments taken into account for determining the number of rearrangements (or “LSTs”).

By a “therapeutically effective amount” of an agent which increases the level of deoxyuridine is meant a sufficient amount to treat cancer, at a reasonable benefit/risk ratio applicable to any medical treatment. It will be understood, however, that the total daily usage of an agent which increases the level of deoxyuridine will be decided by the attending physician within the scope of sound medical judgment. The specific therapeutically effective dose for any particular subject in need thereof will depend upon a variety of factors including other cancer predisposition markers, lifestyle-related risk factors and the activity of the specific agent which increases the level of deoxyuridine to be used, the age, body weight, general health, sex and diet of the subject, the time of administration, route of administration, the duration of the treatment; drugs used in combination or coincidental with the and like factors well known in the medical arts.

The invention also relates to a pharmaceutical composition comprising a PARP inhibitor and/or an alkylating agent for use in a method of treating cancer in a patient, wherein said cancer is linked to deficiency in the HR pathway.

In the pharmaceutical compositions of the present invention for oral, sublingual, subcutaneous, intramuscular, intravenous, transdermal, local or mucosal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and oral administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

In one embodiment, the PARP inhibitor and/or alkylating agent is administered in combination with another active agent.

Typically, the PARP inhibitor and the other active agent can be formulated separately. Alternatively, they can be formulated together in a pharmaceutical composition.

In one embodiment, the PARP inhibitor and/or alkylating agent is administered to a patient who is subjected to radiation therapy and/or surgery in order to remove the tumor.

The invention will be further described by the following examples and figures, which are not intended to limit the scope of the protection defined by the claims.

### FIGURE LEGENDS

FIG. 1. Chromosome content and BRCA1 status in BLCs. A. Distribution of the chromosome content in the set of BLCs displayed two modes, which evidences 2 populations of tumors with different ploidy status. B. Near-diploid tumors (<50 chromosomes) and over-diploid tumors (>=50 chromosomes) showed different proportions of proven BRCA1-inactivated tumors.

WT correspond to non BRCA1.

FIG. 2. Genomic instability in over-diploid BLCs as estimated by the total number of breaks and by LSTs. LST number clearly discriminated non-BRCA1 BLCs from BLCs with proven BRCA1 inactivation (p-value<0.001, Wilcoxon test). Total number of breaks was less significantly different between non-BRCA1 vs BRCA1 and meBRCA1 comparison (p-value<0.03, Wilcoxon test) and was not discriminative. BRCA1: germline BRCA1 mutation; meBRCA1: BRCA1 promoter methylation; sporadic=non-BRCA1: absence of evidence of BRCA1 inactivation.

FIG. 3. Tumor ploidy and the number of large-scale transitions (LST) are discriminative of BRCA1 inactivation in the experimental (left) and validation (right) sets. Upper panel: number of LSTs per tumor is indicated in relation to ploidy categories. Near-diploid and near-tetraploid cutoffs are indicated by a bar. Known BRCA1 and BRCA2 statuses are indicated for germline mutations (“BRCA1” and “BRCA2”), methylation of the BRCA1 promoter (“meBRCA1”) and mutations in the tumors (“tumBRCA1”). Tumors without evidence of BRCA1/2 inactivation are referred to as “non-BRCA1”. Fisher's exact tests are indicated below the contingency tables; BRCA1 refers to all proven BRCA1-inactivated BLCs, non-BRCA1 refers to BLCs without evidence of BRCA1 inactivation.

FIG. 4. Genomic and functional assessments of BRCAness in basal-like cell lines. A. Cell lines with basal-like phenotype display discriminative features of BRCAness similar to primary BLCs. Known status for BRCA1 and BRCA2 are indicated for germline mutations (“BRCA1” and “BRCA2”) and methylation of BRCA1 promoter (“meBRCA1”). Cell lines without evidence of BRCA1/2 inactivation are described as “non-BRCA1/2”. B. RAD51 foci formation 8 hours after 10 Gy irradiation illustrates active homologous recombination (HR) in non-BRCA1 cell lines, and conversely deficient HR in BRCA1 or BRCA2 mutated cell lines. 53BP1 foci in the same experiment are shown as a control for DNA damage response. Scale bars, 20 μm). Number of LST is indicated as well as BRCA1/2 status: mut, mutated; me, methylation of the promoter; wt, wildtype.

FIG. 5. Survival curves for LST_high and LST_low ovarian tumors. P-value was estimated by log-rank test statistic.

FIG. 6. Event free survival curves for LST_high and LST_low ovarian tumors. P-value was estimated by log-rank test statistic.

FIG. 7. LST_10 Mb in tumor cell lines.

Calculated ploidy is indicated (2N pseudo-diploid, 4N pseudo-tetraploid). Triangle: wild-type or unknown BRCA1/2 status; square: BRCA2 mutated cell lines.

### EXAMPLES

### Example 1

### Materials and Methods

### Patients and Tumors

A series of undifferentiated grade 3 BLCs was assembled from patients who had surgery at the Institut Curie. According to French regulations patients were informed of research and did not express opposition. High quality biological material was available at Institut Curie biobank for 85 tumor samples (some samples were described previously).²⁸⁻³⁰ This series was enriched for tumors arisen in patients carrying deleterious BRCA1 mutations (35 tumors).

### Immunohistochemistry

Immunostaining was performed on 4 μm tissue sections as described previously:^{28,29} ER, PR and ERBB2 (Novocastra), EGFR and KRT8/18 (Zymed, Invitrogen), KRT5/6 (Dako) and KRT14 (Biogenex). Positivity for each marker was determined according to standardized guidelines.³¹ Negativity was defined as total absence of staining for expression of ER and PR, and as less than 2+ staining for ERBB2.

The basal-like phenotype was defined according to morphological, phenotypic and/or molecular criteria including i) high grade (Elston-Ellis grading) and pushing margins, ii) triple-negative phenotype and expression of either KRT5/6/14/17 or EGFR assessed by immunohistochemistry.³²

### Methylation Status of BRCA1 Promoter

Methylation of the promoter of BRCA1 was assessed by methyl-specific PCR (MSP) after bisulfite conversion as described previously,³³ with minor modifications (primer sequences and protocols are available upon request).

### BRCA1 Mutation Status

Pre-screen for mutations of the BRCA1 gene was performed using Enhanced Mismatch Mutation Analysis (EMMA, Fluigent³⁴; EMMALYS software P/N: 5331254102). For abnormal EMMA profiles, the concerned BRCA1 exons were sequenced with dideoxynucleotides (BigDye Terminator V1.1, Applied Biosystems, Foster City, CA), according to standard protocols (primer sequences and protocols are available upon request). Sequences were examined with the Seqscape V2.5 (Applied Biosystems).

### Analysis of Transcriptomic Data

Transcriptomic data was obtained on the Affymetrix U133plus2 platform in the Institut Curie according to the standard protocol. Normalization was performed with BrainArray algorithm³⁵. Unsupervised clustering was performed based on the intrinsic signature³⁶.

### Processing the Genomic Profiles

Genomic profiling of 85 BLCs was performed using two platforms: Illumina (Illumina SNP HapMap 300K Duo, 33 cases) and Affymetrix (Affymetrix SNP Chip 6.0, 52 cases).

Illumina platform: Genomic profiling of the tumor samples was performed by a service provider (Integragen, Evry, France) on 300K Illumina SNP-arrays (Human Hap300-Duo). Raw data files were processed by BeadStudio 3.3 in standard settings using supporting data provided by Illumina (HumanHap300v2_A). Allele specific signals (X and Y in BeadStudio notation) were processed into Log R ratio and B allele frequency by tQN algorithm.³⁷

Affymetrix platform: Hybridization was performed at Institut Curie on Affymetrix SNPChip6.0 array. Cell files were processed by Genotyping Console 3.0.2. Log 2Ratio and Allele Difference profiles resulted from Copy Number and LOH analysis performed with the reference model file HapMap270 (GenomeWideSNP_6.hapmap270.na29) provided by Affymetrix.

Quality control: 20 SNP arrays were discarded due to: low hybridization quality (3 arrays); low tumor content and/or ambiguous profile interpretation (17 arrays).

Segmental copy number and genotype detection: Both Illumina and Affymetrix SNP array data were mined using the GAP method described and validated previously: segmental copy numbers, allelic contents (major allele counts) and normal cell contamination were detected; segmentations were optimized with respect to the genomic status detected.²⁷

Recognition of absolute copy number ranged from 0 to 8 copies with all segments exceeding 8-copy level been ascribed 8-copy status. Thus, 22 possible segmental genotypes were discriminated (copy number/major allele count): 1 copy A (or 1/1); 2 copies AA (2/2) and AB (2/1); 3 copies AAA (3/3), AAB (3/2); 4 copies AAAA (4/4), AAAB (4/3), AABB (4/2), etc. . . . .

Chromosome number: Number of chromosomes was estimated by the sum of the copy numbers detected at the pericentric regions. The status of the pericentric region of each chromosome arm was defined by the corresponding juxta-centromeric segment when the latter contained 500 SNPs or more. When not measurable, missing values were substituted by the modal copy number of the considered chromosome arm (3.4±2.2 out of 41 chromosome arms per genome were substituted in the series). Chromosome counting procedure was validated by comparing estimated chromosome numbers versus available numbers from karyotype or SKY data for 25 breast cancer cell lines {lgcstandards-atcc.org/}. Error rate was less than 2 chromosomes per sample (1.58±2.3).

Breakpoint counts: Number of breakpoints in each genomic profile was estimated based on the resulting interpretable copy number profile and after filtering less than 50 SNPs variation. Small interstitial alterations were defined as <3 Mb alterations surrounded by the segments with identical status for genotype and copy number. They were removed when estimating total number of breakpoints. Large-scale State Transitions (LSTs) were calculated after smoothing and filtering of variation less than 3 Mb in size.

### Compilation of Validation Sets

The validation series comprises 55 samples including TNBC from a cohort of young women with breast cancer (17 cases); BLCs with medullary features (8 cases) and one BLC arisen in a BRCA2 mutation carrier; BRCA1 BLCs from GEO GSE19177 (12 cases)³⁸; basal-like tumors from GEO GSE32530 (4 cases)³⁹; BRCA1 BLCs from Institut Bergonié (5 cases).

Basal-like cell lines with available SNP array profile comprised 17 cases (15 cases hybridized in Institute Curie and 2 cases were obtained from the Wellcome Trust Sanger Institute Cancer Genome Project web site.

### Results

### BRCA1 Status of Basal-Like Carcinomas (BLCs)

A series of 65 well characterized basal-like breast carcinomas included 23 tumors arisen in patients carrying deleterious BRCA1 mutations (herein called “BRCA1 BLCs”) and 42 BLCs arisen in patients without evidence of familial predisposition of breast/ovarian cancer or tested negative for BRCA1/2 mutations (herein called “sporadic BLCs”). Sporadic BLCs were tested for the methylation of the BRCA1 promoter and nearly 25% were found positive (11 out of 41 tested, herein called “meBRCA1 BLCs”). No evidence of methylation in the remaining 31 cases was found. BRCA status was confirmed by the gene expression in 35 out of 36 tested cases with available transcriptomic data. BRCA1 and meBRCA1 BLCs comprise the group of tumors with proven BRCA1 inactivation (34 cases), which were further compared to the group of presumably non-BRCA1 BLCs (31 cases).

### Near-Diploidy in BLCs has 75% Positive Predictive Value of BRCA1 Inactivation

In order to get insight into the specific genomic alterations of BLCs, genomic profiling was performed using SNP-arrays, which provide two complementary measurements: copy number variation and allelic imbalance. Genome Alteration Print (GAP) methodology for mining SNP arrays²⁷ allowed us to obtain the segmental genotype profiles (i.e. exact copy numbers and allelic contents: A, AB, AA, AAB, AAA, . . . ) for each sample. General genomic characteristics such as number of chromosomes, DNA index, number of chromosome breaks, and proportions of genome in each genomic state were inferred from the segmental genotype profiles.

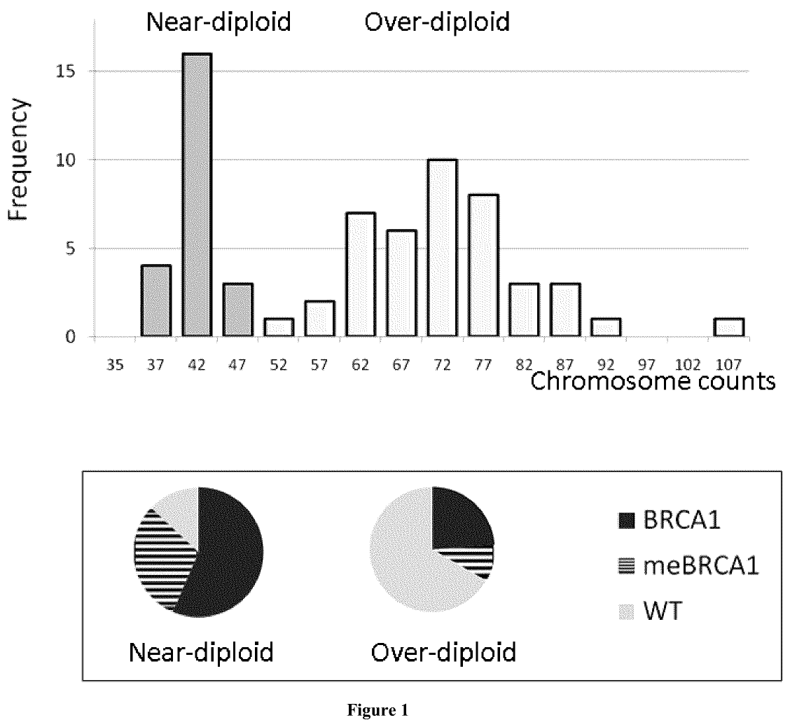

Estimated chromosome counts per genome showed a bimodal distribution (FIG. 1, top panel) similar to those demonstrated for the genomes in various types of cancers⁴⁰. Tumor genomes carrying less than 50 chromosomes and with the DNA index close to 1 were considered to have ploidy of two and were thereafter called “near-diploid genomes” (23 cases). Following the hypothesis of the whole genome duplication during cancer progression explaining the second mode in chromosome distribution⁴⁰ tumor genomes carrying more than 50 chromosomes and DNA index higher than 1.2 were considered to have a ploidy of four and were thereafter called “over-diploid genomes” (42 cases).

Interestingly, the 23 near-diploid tumors almost consistently carried germline mutation or epigenetic inactivation of BRCA1 (20/23) in contrast to the over-diploid tumors, which were slightly enriched in non-BRCA1 BLCs (28/42) (FIG. 1, bottom pannel). Taking into account the fact that BRCA1 germline mutation is responsible for near 10% of basal-like carcinomas⁴¹ positive predictive value of genomic near-diploid status was estimated to be 75%.

### Large-Scale Chromosomal Rearrangements Discriminate BRCA1 and Non-BRCA1 Basal-Like Carcinomas

Total number of breakpoints detected in the cancer genome characterizes the level of genomic instability. However, overall comparison of BRCA1 versus non-BRCA1 tumors did not show any significant difference (p-value=0.28). In the subgroup of 42 over-diploid BLCs, 14 BRCA-inactivated tumors displayed elevated total number of breakpoints (range [57-224], 140.6±45.7), while 28 non-BRCA1 tumors showed significant heterogeneity (range [8-213], 101.2±50.6) and were enriched in the low values compared to BRCA1 tumors (p<0.017, Wilcoxon rank test). However, large overlap in the breakpoint numbers precluded accurate demarcation. In order to get a robust and discriminative estimation of the genomic instability we evaluated the number of Large-scale State Transitions (LSTs) by calculating chromosomal breaks between adjacent regions of at least 10 Mb (comprising ˜3000 SNPs in Affymetrix SNP6.0).

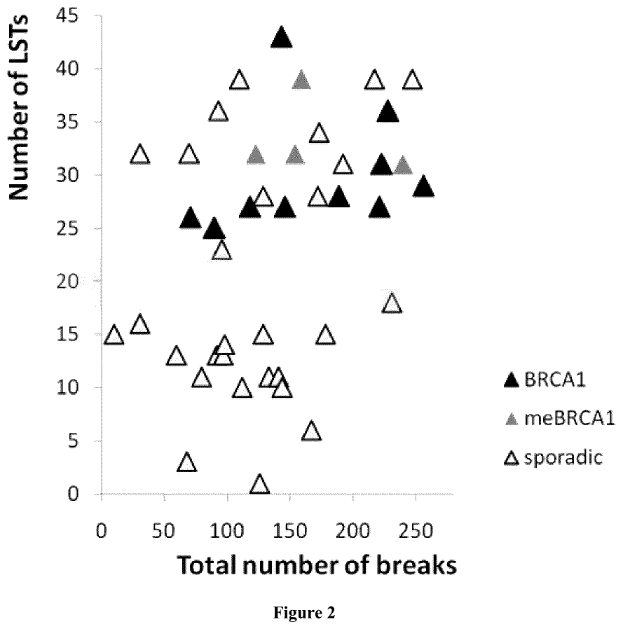

Number of LSTs in the subgroup of over-diploid tumors had a bimodal distribution with a clear gap between two modes (12.5±4.9 and 35.5±6.7) separating 18 non-BRCA1 BLCs from the mixture containing 14 BRCA1-inactivated tumors and 10 tumors with neither BRCA1 germline mutation nor BRCA1 promoter methylation (FIG. 2). In the subgroup of 23 near-diploid BLCs, which mainly contained BRCA1 tumors, LSTs had unimodal distribution (28.0±6.5) with two non-BRCA1 tumors within one standard deviation (24 and 28 LSTs) and one non-BRCA1 BLC below two standard deviations from the average (12 LSTs). Interestingly, all tumors with low LSTs had no evidence of BRCA1 inactivation and displayed either few chromosomal breaks and a high level of aneuploidy (3 samples) or firestorm-like alterations (16 samples).

To conclude, LSTs reflected well the overall genomic patterns of the tumors, contrary to the total number of breakpoints, and provided the discriminative values for BRCA1 status prediction.

### A Two-Step Decision Rule Consistently Detects BRCA1 Inactivation in BLCs.

Based on the LSTs distributions described above, two thresholds for BRCAness prediction were applied, more than 15 LSTs per genome in the near-diploid cases and more than 20 LSTs in the over-diploid cases, predicting BRCAness with 100% sensitivity (p-value=4*10⁻⁵, Fisher test).

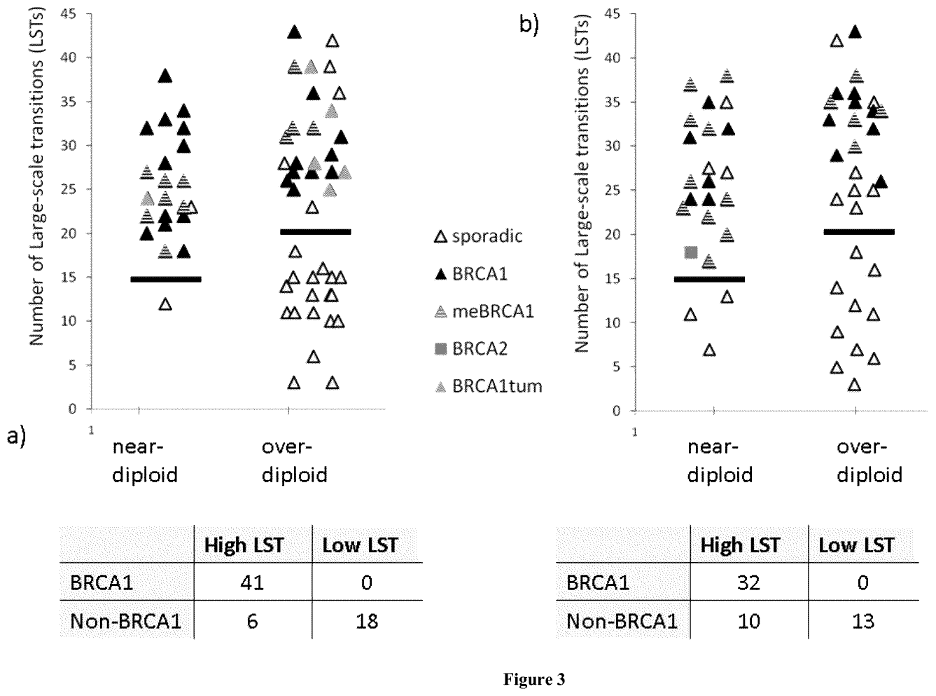

Moreover, all “False Positive” cases (thereafter called “BRCA1-looking” BLCs) had similar high number of LSTs as the “True Positive” cases (with proven BRCA1-inactivated status), which actually questioned their false positive status and might evidence other mechanisms of homologous recombinaison defect including BRCA1 or BRCA2 mutations. Such mutations were searched in 28 sporadic BLCs with available material including 13 cases with the BRCA1-looking pattern. Deleterious BRCA1 mutations were found in six cases all belonging to BRCA1-looking tumors (p-value=0.02). Deleterious BRCA2 mutations were found in three cases all belonging to BRCA1-looking tumors. With these findings specificity reached 89% (p-value=1.4*10⁻¹¹, Fisher test) in the considered experimental set of BLCs (FIG. 3A).

A validation series of 55 BLC/TNBC was assembled, including 15 cases with BRCA1 germline mutations, 15 cases with BRCA1 promoter methylation, 1 case with a BRCA2 germline mutation, and 24 sporadic cases. SNP array data were processed using the same workflow. Prediction of the BRCA1 inactivation displayed sensitivity of 100% (all 30 BRCA1 inactivated cases were predicted to be BRCA1-looking) and specificity of 80% (11 cases were predicted to be BRCA1-looking with yet no evidence of BRCA1 inactivation) (FIG. 3B; p-value=1.7*10⁻⁶, Fisher test). Noteworthy, the BRCA2 mutated tumor was near-diploid with a high LST number, thus clearly following a BRCA1-looking pattern.

### Model Systems Supported the Discriminative Features Observed in the Primary Tumors

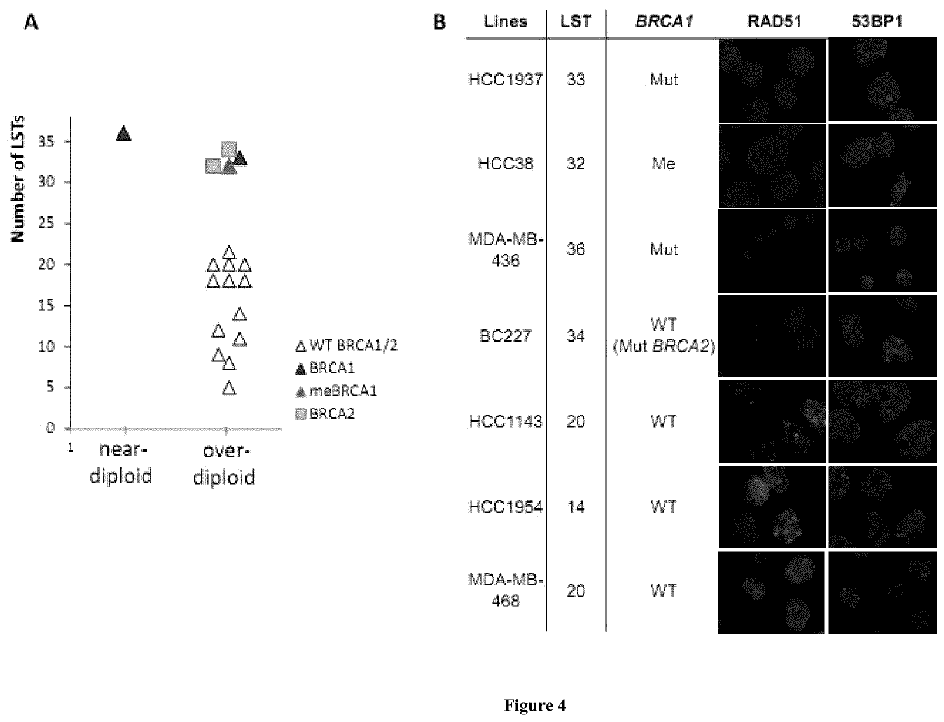

A series of 17 basal-like cell lines was analyzed, including MDA-MB-436 and HCC1937 bearing BRCA1 mutations⁴² and HCC38 with BRCA1 promoter methylation⁴³. The obtained results followed the trend found in primary tumors: firstly the only near-diploid cell line found was the BRCA1 mutated MDA-MB-436; secondly among over-diploid cell lines, HCC1937 and HCC38 carried the highest number of large-scale chromosomal breaks, which is again consistent with their BRCA1-inactivated status. Nevertheless, and as expected considering cell line establishment and long term maintenance in culture, the cutoff separating non-BRCA1 cell lines was found shifted to 23 LSTs (FIG. 4). One cell line HCC1599 had LST number very close to BRCA1 inactivated cell lines, whereas not associated with BRCA1/2 mutation⁴⁴. To clarify the BRCA1 function and more precisely the homologous recombination pathway, RAD51 foci were measured 8 hours after ionizing radiations in BLC cell lines. All cell lines without BRCA1 looking pattern had the expected RAD51 foci accumulation, whereas no foci were observed in cell lines with BRCA1 looking pattern, including HCC1599 (data not shown).

In conclusion, the inventors have shown that it is possible to predict tumor deficiency in the DNA homologous recombination (HR) pathway in a patient suffering from cancer, by quantifying the number of rearrangements in the genomic DNA of a tumor sample obtained from said patient, wherein the number of rearrangements corresponds to the number, per genome, of breakpoints resulting in segments of at least 10 megabases.

Similar results were obtained by using a cutoff value between 3 megabases and 20 megabases for the definition of Large Scale Transitions.

### Example 2—Performance of LST Number Predicting BRCAness in all Types of Breast Carcinomas

The series of 426 breast tumors (invasive ductal carcinomas including HER2-positive tumors, luminal (eg expressing receptors for estrogen or progesterone), triple negative/basal-like breast carcinoma (eg expressing no hormone receptors and not overexpressing HER2) as well as rare subtypes such as medullary carcinomas or micropapillary carcinomas from Institut Curie) was considered. The series was enriched with BRCA1 and BRCA2 mutated tumors. The cut-offs on the LST number predicting BRCAness were inferred based on this series (Table 1). False Positive and True Positive Rates (FPR and TPR) show the quality of LST based predictor of BRCAness.

TABLE 1
Cut-offs for breast cancer BRCAness prediction based on the LST
number

| LST_S | Ploidy 2: (P = 68, N = 182) | | | Ploidy 4: (P = 53, N = 123) | | |
|---|---|---|---|---|---|---|
| Mb, S | Cut-Off* | FPR | TPR | Cut-Off | FPR | TPR |
| 6 | 19 (17) | 0.04 | 0.99 | 32 (32) | 0.10 | 1 |
| 7 | 17 (15) | 0.05 | 0.99 | 29 (27) | 0.07 | 0.98 |
| 8 | 14 (14) | 0.06 | 1 | 26 (26) | 0.08 | 1 |
| 9 | 14 (11) | 0.04 | 0.99 | 25 (19) | 0.07 | 0.98 |
| 10 | 11 (11) | 0.07 | 1 | 22 (18) | 0.06 | 0.98 |

*Cut-offs correspond to max(TPR-FPR);
cut-offs in parenthesis correspond to 100 sensitivity.
P: Number of positives, i.e. BRCA1/2 mutated tumors;
N: Number of negatives, i.e. number of tumors with BRCA1/2 wild-type or status not available;
TPR: True positive rate;
FPR: False positive rate.

### Example 3—the Number of LSTs is a Good Predictor of Response to Treatment

Two publically available data sets from clinical trial of Cisplatin treatment of patients with triple-negative breast tumors [GSE28330 GEO database][59] were processed and the number of LST_10 Mb was calculated for each tumor with good quality of measured profile. Genomic profiles were measured by two types of chip: Affymetrix Oncoscan 70K (Dataset 2) and Oncoscan 300K (Dataset 1). Information about mutational status of BRCA1/2 was available for some tumors. Response to treatment was measured by Miller-Payne score, where 4 and 5 were considered as “positive response”, while scores<4 were considered as “no response” Case by case and summary results are presented in Table 2 and Tables 3-5 (statistical comparisons were performed by the Fisher exact test). To conclude, (i) almost all known BRCA1/2 inactivated cases (17/18) and 15 tumors with wild-type or unknown BRCA1/2 status were classified as LST_high (Table 3); (ii) BRCA1/2 inactivation does not always mean response to Cisplatin (Table 4); (iii) LST_10 Mb is a better cisplatin response predictor than the BRCA1/2 status (Table 4-5).

TABLE 2
Individual results

| | | | | Miller- | | |
|---|---|---|---|---|---|---|
| Data | | Recognition | | Payne | | |
| set | ID | Quality | BRCA1/2 | response | LST | Response |
| 1 | DFHCC_06.202_45R | good | | 5 | High | Yes |
| 1 | DFHCC_06.202_15 | good | mut | 5 | High | Yes |
| 1 | DFHCC_06.202_41 | good | | 5 | High | Yes |
| 1 | DFHCC_06.202_7 | good | mut | 5 | High | Yes |
| 1 | DFHCC_06.202_17 | good | | 5 | High | Yes |
| 2 | DFHCC_04.183_9T | good | non | 5 | High | Yes |
| 2 | DFHCC_04.183_18T | good | mut | 5 | High | Yes |
| 2 | DFHCC_04.183_3T | good | non | 5 | High | Yes |
| 2 | DFHCC_04.183_29T | good | non | 5 | High | Yes |
| 2 | DFHCC_04.183_5T | good | mut | 5 | High | Yes |
| 2 | DFHCC_04.183_17T | good | met | 5 | High | Yes |
| 1 | DFHCC_06.202_6 | good | met | 4 | High | Yes |
| 1 | DFHCC_06.202_48 | good | met | 4 | High | Yes |
| 2 | DFHCC_04.183_7T | good | met | 4 | High | Yes |
| 2 | DFHCC_04.183_8T | good | met | 4 | High | Yes |
| 1 | DFHCC_06.202_40 | good | | 4 | High | Yes |
| 2 | DFHCC_04.183_10T | good | non | 4 | High | Yes |
| 1 | DFHCC_06.202_3 | good | | 4 | High | Yes |
| 1 | DFHCC_06.202_27 | good | | 4 | Low | Yes |
| 1 | DFHCC_06.202_13 | good | met | 3 | High | No |
| 1 | DFHCC_06.202_5 | good | | 3 | Low | No |
| 1 | DFHCC_06.202_4 | good | met | 3 | High | No |
| 2 | DFHCC_04.183_23T | good | met | 3 | High | No |
| 2 | DFHCC_04.183_11T | good | non | 3 | High | No |
| 2 | DFHCC_04.183_25T | good | met | 3 | High | No |
| 2 | DFHCC_04.183_1T | good | met | 3 | High | No |
| 1 | DFHCC_06.202_37 | good | | 3 | Low | No |
| 1 | DFHCC_06.202_20 | good | mut | 2 | High | No |
| 1 | DFHCC_06.202_42 | good | mut | 2 | High | No |
| 1 | DFHCC_06.202_21 | good | | 2 | High | No |
| 2 | DFHCC_04.183_14T | good | non | 2 | High | No |
| 2 | DFHCC_04.183_24T | good | non | 2 | Low | No |
| 2 | DFHCC_04.183_22T | good | non | 2 | Low | No |
| 2 | DFHCC_04.183_28T | good | non | 2 | Low | No |
| 1 | DFHCC_06.202_24 | good | | 2 | Low | No |
| 1 | DFHCC_06.202_10 | good | | 1 | Low | No |
| 1 | DFHCC_06.202_32 | good | | 1 | Low | No |
| 1 | DFHCC_06.202_35 | good | | 1 | Low | No |
| 1 | DFHCC_06.202_46 | good | | 1 | Low | No |
| 2 | DFHCC_04.183_13T | good | non | 1 | Low | No |
| 1 | DFHCC_06.202_34 | good | | 1 | High | No |
| 1 | DFHCC_06.202_29 | good | | 1 | High | No |
| 1 | DFHCC_06.202_45L | good | | 1 | High | No |
| 2 | DFHCC_04.183_4T | good | non | 1 | High | No |
| 2 | DFHCC_04.183_12T | good | non | 1 | Low | No |
| 1 | DFHCC_06.202_18 | good | | 1 | Low | No |
| 1 | DFHCC_06.202_9 | good | | 1 | Low | No |
| 2 | DFHCC_04.183_16T | good | non | 1 | Low | No |
| 1 | DFHCC_06.202_14 | good | | 1 | Low | No |
| 2 | DFHCC_04.183_6T | good | | 1 | Low | No |
| 1 | DFHCC_06.202_28 | good | | 0 | Low | No |
| 2 | DFHCC_04.183_21T | good | non | 0 | High | No |
| 2 | DFHCC_04.183_27T | good | non | 0 | Low | No |
| 2 | DFHCC_04.183_26T | good | met | 0 | Low | No |
| 2 | DFHCC_04.183_15T | bad | met | 0 | | No |
| 2 | DFHCC_04.183_20T | bad | non | 2 | | No |
| 2 | DFHCC_06.202_33 | good | | NA | | |
| 2 | DFHCC_06.202_43 | good | | NA | | |
| 2 | DFHCC_06.202_50 | good | | NA | | |
| 2 | DFHCC_06.202_39 | bad | | 2 | | No |
| 2 | DFHCC_06.202_39 | bad | | 2 | | No |

TABLE 3
Summary of LST versus BRCA1/2

| ALL | LST_high | LST_Iow | |
|---|---|---|---|
| BRCA1/2 | 18 | 1 | p < 0.0001 |
| NON BRCA1/2 or NA | 15 | 20 | |

TABLE 4
Summary of BRCA1/2 versus Response

| ALL | Responders | Non Responders | |
|---|---|---|---|
| BRCA1/2 | 9 | 8 | p < 0.06 |
| NON BRCA1/2 or NA | 10 | 27 | |

TABLE 5
Summary of LST versus Response

| ALL | LST_high | LST_Iow | |
|---|---|---|---|
| Non Responders | 15 | 20 | p < 0.0001 |
| Responders | 18 | 1 | |

### Example 4—LST in Ovarian Carcinoma

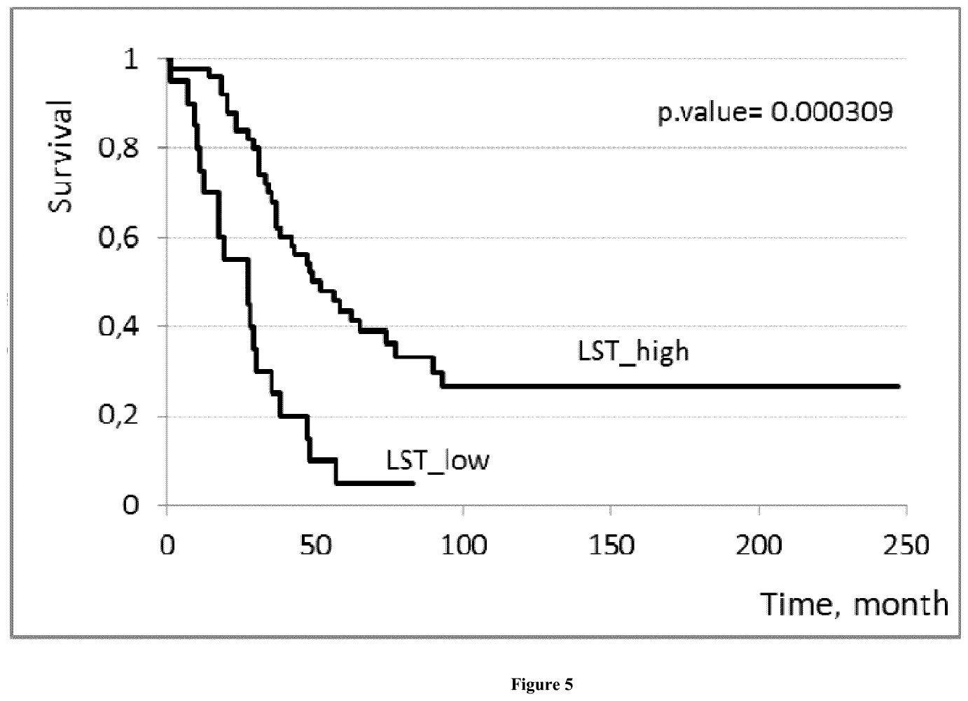

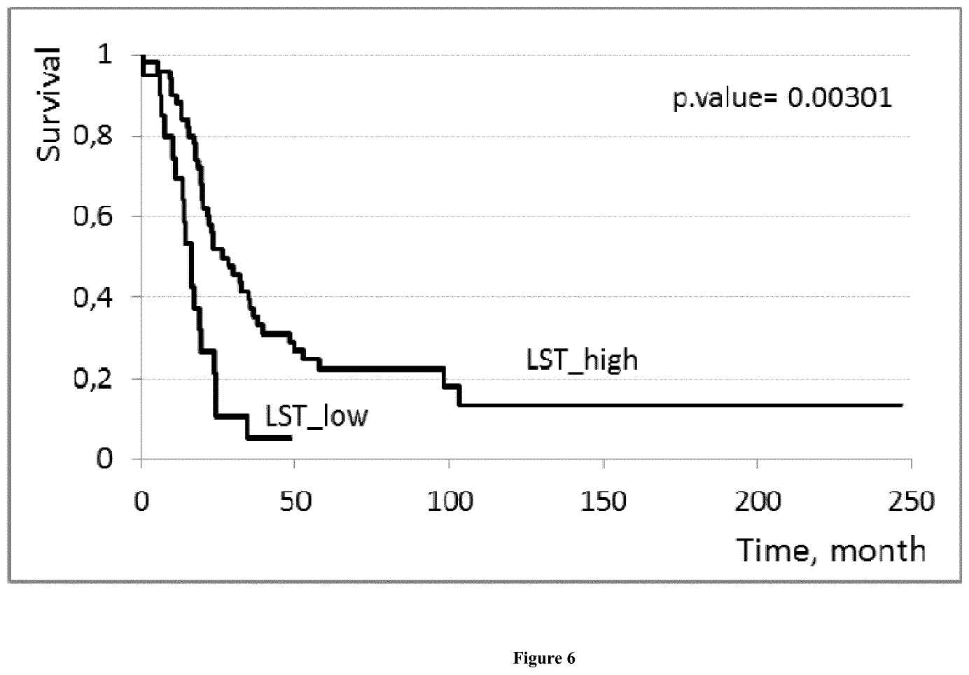

Series of high grade ovarian carcinoma from Institut Curie were profiled by SNP arrays (Affymetrix CytoScanHD). All patients were treated by chemotherapies including platinum salts. Tumor genomes were annotated as LST_high (50 cases) and LST_low (20 cases) based on the LST_6 Mb with the cutoffs 19 and 32 LSTs for near-diploid and near-tetraploid tumors respectively. Comparison of Overall Survival and Event Free Survival showed better outcome for patients with LST_high tumors, which indicates better response to treatment (FIGS. 5-6).

### Example 5—LST in Tumor Cell Lines

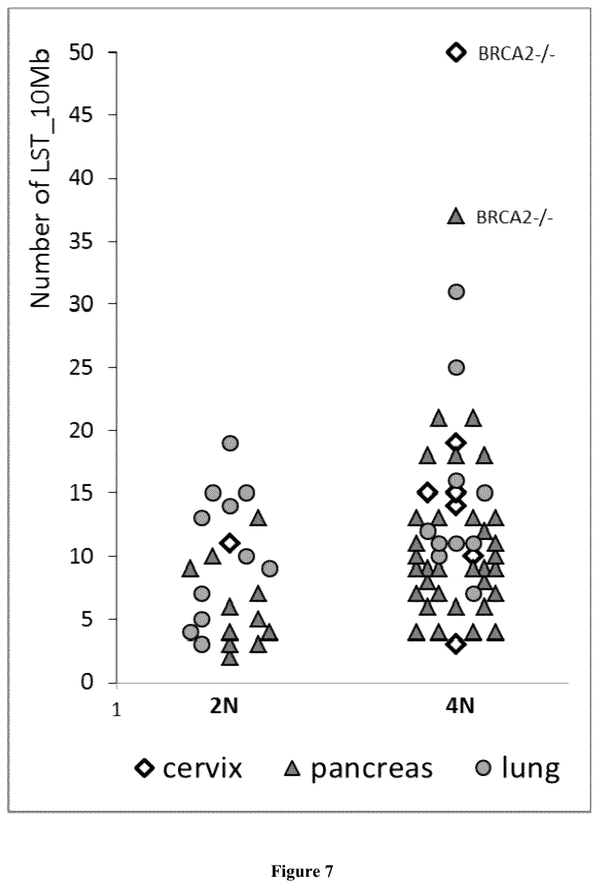

Series of tumor cell lines with known BRCA status and with available SNP-array data were analyzed. LST_10 Mb was calculated and samples with high LST were linked to BRCA2 inactivation in cervix and pancreatic carcinoma cell lines. Two lung cell lines without known BRCA1/2 mutations have a high level of LST, presumably due to BRCA1 methylation described in this disease (FIG. 7).

This validation of the method in tumor cell lines of various origins and state of differentiation indicates that LST measurement and prediction of the BRCAness can be applied in all types of tumors.

### REFERENCES

ADDIN EN.REFLIST 1. Rakha E A, Reis-Filho J S, Ellis I O: Basal-like breast cancer: a critical review. J Clin Oncol 26:2568-81, 2008
2. Dawson S J, Provenzano E, Caldas C: Triple negative breast cancers: clinical and prognostic implications. Eur J Cancer 45 Suppl 1:27-40, 2009
3. Foulkes W D, Stefansson I M, Chappuis P O, et al: Germline BRCA1 mutations and a basal epithelial phenotype in breast cancer. J Natl Cancer Inst 95:1482-5, 2003
4. Bergamaschi A, Kim Y H, Wang P, et al: Distinct patterns of DNA copy number alteration are associated with different clinicopathological features and gene-expression subtypes of breast cancer. Genes Chromosomes Cancer 45:1033-40, 2006
5. Melchor L, Honrado E, Garcia M J, et al: Distinct genomic aberration patterns are found in familial breast cancer associated with different immunohistochemical subtypes. Oncogene 27:3165-75, 2008
6. Natrajan R, Weigelt B, Mackay A, et al: An integrative genomic and transcriptomic analysis reveals molecular pathways and networks regulated by copy number aberrations in basal-like, HER2 and luminal cancers. Breast Cancer Res Treat, 2009
7. Gudmundsdottir K, Ashworth A: The roles of BRCA1 and BRCA2 and associated proteins in the maintenance of genomic stability. Oncogene 25:5864-74, 2006
8. Roy R, Chun J, Powell S N: BRCA1 and BRCA2: different roles in a common pathway of genome protection. Nat Rev Cancer 12:68-78, 2012
9. Turner N, Tutt A, Ashworth A: Hallmarks of ‘BRCAness’ in sporadic cancers. Nat Rev Cancer 4:814-9, 2004
10. Chin S F, Teschendorff A E, Marioni J C, et al: High-resolution aCGH and expression profiling identifies a novel genomic subtype of E R negative breast cancer. Genome Biol 8:R215, 2007
11. Stefansson O A, Jonasson J G, Johannsson O T, et al: Genomic profiling of breast tumours in relation to BRCA abnormalities and phenotypes. Breast Cancer Res 11:R47, 2009
12. Joosse S A, Brandwijk K I, Mulder L, et al: Genomic signature of BRCA1 deficiency in sporadic basal-like breast tumors. Genes Chromosomes Cancer 50:71-81, 2011
13. Farmer H, McCabe N, Lord C J, et al: Targeting the DNA repair defect in BRCA mutant cells as a therapeutic strategy. Nature 434:917-21, 2005
14. Bryant H E, Schultz N, Thomas H D, et al: Specific killing of BRCA2-deficient tumours with inhibitors of poly(ADP-ribose) polymerase. Nature 434:913-7, 2005
15. Fong P C, Boss D S, Yap T A, et al: Inhibition of poly(ADP-ribose) polymerase in tumors from BRCA mutation carriers. N Engl J Med 361:123-34, 2009
16. Vollebergh M A, Jonkers J, Linn S C: Genomic instability in breast and ovarian cancers: translation into clinical predictive biomarkers. Cell Mol Life Sci 69:223-45, 2012
17. Focken T, Steinemann D, Skawran B, et al: Human BRCA1-Associated Breast Cancer: No Increase in Numerical Chromosomal Instability Compared to Sporadic Tumors. Cytogenet Genome Res 135:84-92, 2011
18. Johannsdottir H K, Jonsson G, Johannesdottir G, et al: Chromosome 5 imbalance mapping in breast tumors from BRCA1 and BRCA2 mutation carriers and sporadic breast tumors. Int J Cancer 119:1052-60, 2006
19. Tirkkonen M, Johannsson O, Agnarsson B A, et al: Distinct somatic genetic changes associated with tumor progression in carriers of BRCA1 and BRCA2 germ-line mutations. Cancer Res 57:1222-7, 1997
20. Melchor L, Alvarez S, Honrado E, et al: The accumulation of specific amplifications characterizes two different genomic pathways of evolution of familial breast tumors. Clin Cancer Res 11:8577-84, 2005
21. Wessels L F, van Welsem T, Hart A A, et al: Molecular classification of breast carcinomas by comparative genomic hybridization: a specific somatic genetic profile for BRCA1 tumors. Cancer Res 62:7110-7, 2002
22. Waddell N, Arnold J, Cocciardi S, et al: Subtypes of familial breast tumours revealed by expression and copy number profiling. Breast Cancer

### Res Treat, 2009

23. Jonsson G, Staaf J, Vallon-Christersson J, et al: Genomic subtypes of breast cancer identified by array-comparative genomic hybridization display distinct molecular and clinical characteristics. Breast Cancer Res 12:R42, 2010
24. Joosse S A, van Beers E H, Tielen I H, et al: Prediction of BRCA1-association in hereditary non-BRCA1/2 breast carcinomas with array-CGH. Breast Cancer Res Treat 116:479-89, 2009
25. Lips E H, Mulder L, Hannemann J, et al: Indicators of homologous recombination deficiency in breast cancer and association with response to neoadjuvant chemotherapy. Ann Oncol 22:870-6, 2011
26. Vollebergh M A, Lips E H, Nederlof P M, et al: An aCGH classifier derived from BRCA1-mutated breast cancer and benefit of high-dose platinum-based chemotherapy in HER2-negative breast cancer patients. Ann Oncol 22:1561-70, 2011
27. Popova T, Manie E, Stoppa-Lyonnet D, et al: Genome Alteration Print (GAP): a tool to visualize and mine complex cancer genomic profiles obtained by SNP arrays. Genome Biol 10:R128, 2009
28. Manie E, Vincent-Salomon A, Lehmann-Che J, et al: High frequency of TP53 mutation in BRCA1 and sporadic basal-like carcinomas but not in BRCA1 luminal breast tumors. Cancer Res 69:663-71, 2009
29. Vincent-Salomon A, Gruel N, Lucchesi C, et al: Identification of typical medullary breast carcinoma as a genomic sub-group of basal-like carcinomas, a heterogeneous new molecular entity. Breast Cancer Res 9:R24, 2007
30. Marty B, Maire V, Gravier E, et al: Frequent PTEN genomic alterations and activated phosphatidylinositol 3-kinase pathway in basal-like breast cancer cells. Breast Cancer Res 10:R101, 2008
31. Azoulay S, Lae M, Freneaux P, et al: KIT is highly expressed in adenoid cystic carcinoma of the breast, a basal-like carcinoma associated with a favorable outcome. Mod Pathol 18:1623-31, 2005
32. Nielsen T O, Hsu F D, Jensen K, et al: Immunohistochemical and clinical characterization of the basal-like subtype of invasive breast carcinoma. Clin Cancer Res 10:5367-74, 2004
33. Esteller M, Silva J M, Dominguez G, et al: Promoter hypermethylation and BRCA1 inactivation in sporadic breast and ovarian tumors. J Natl Cancer Inst 92:564-9, 2000
34. Houdayer C, Moncoutier V, Champ J, et al: Enhanced mismatch mutation analysis: simultaneous detection of point mutations and large scale rearrangements by capillary electrophoresis, application to BRCA1 and BRCA2. Methods Mol Biol 653:147-80, 2010
35. Dai M, Wang P, Boyd A D, et al: Evolving gene/transcript definitions significantly alter the interpretation of GeneChip data. Nucleic Acids Res 33:e175, 2005
36. Sorlie T, Tibshirani R, Parker J, et al: Repeated observation of breast tumor subtypes in independent gene expression data sets. Proc Natl Acad Sci USA 100:8418-23, 2003
37. Staaf J, Vallon-Christersson J, Lindgren D, et al: Normalization of Illumina Infinium whole-genome SNP data improves copy number estimates and allelic intensity ratios. BMC Bioinformatics 9:409, 2008
38. Waddell N, Arnold J, Cocciardi S, et al: Subtypes of familial breast tumours revealed by expression and copy number profiling. Breast Cancer Res Treat 123:661-77, 2010
39. DeRose Y S, Wang G, Lin Y C, et al: Tumor grafts derived from women with breast cancer authentically reflect tumor pathology, growth, metastasis and disease outcomes. Nat Med 17:1514-20, 2011
40. Storchova Z, Kuffer C: The consequences of tetraploidy and aneuploidy. J Cell Sci 121:3859-66, 2008
41. Young S R, Pilarski R T, Donenberg T, et al: The prevalence of BRCA1 mutations among young women with triple-negative breast cancer. BMC Cancer 9:86, 2009
42. Elstrodt F, Hollestelle A, Nagel J H, et al: BRCA1 mutation analysis of 41 human breast cancer cell lines reveals three new deleterious mutants.

### Cancer Res 66:41-5, 2006

43. Xu J, Huo D, Chen Y, et al: CpG island methylation affects accessibility of the proximal BRCA1 promoter to transcription factors. Breast Cancer Res Treat 120:593-601, 2010
44. Sjoblom T, Jones S, Wood L D, et al: The consensus coding sequences of human breast and colorectal cancers. Science 314:268-74, 2006
45. Garcia A I, Buisson M, Bertrand P, et al: Down-regulation of BRCA1 expression by miR-146a and miR-146b-5p in triple negative sporadic breast cancers. EMBO Mol Med 3:279-90, 2011
46. Moskwa P, Buffa F M, Pan Y, et al: miR-182-mediated downregulation of BRCA1 impacts DNA repair and sensitivity to PARP inhibitors. Mol Cell 41:210-20, 2011
47. Plo I, Laulier C, Gauthier L, et al: AKTI inhibits homologous recombination by inducing cytoplasmic retention of BRCA1 and RAD51. Cancer Res 68:9404-12, 2008
48. Van Loo P, Nordgard S H, Lingjaerde O C, et al: Allele-specific copy number analysis of tumors. Proc Natl Acad Sci USA 107:16910-5, 2010
49. Pujana M A, Han J D, Starita L M, et al: Network modeling links breast cancer susceptibility and centrosome dysfunction. Nat Genet 39:1338-49, 2007
50. Xu X, Weaver Z, Linke S P, et al: Centrosome amplification and a defective G2-M cell cycle checkpoint induce genetic instability in BRCA1 exon 11 isoform-deficient cells. Mol Cell 3:389-95, 1999
51. Brodie K M, Henderson B R: Characterization of BRCA1 centrosome targeting, dynamics and function: A role for the nuclear export signal, CRM1 and Aurora A kinase. J Biol Chem, 2012
52. Kais Z, Parvin J D: Regulation of centrosomes by the BRCA1-dependent ubiquitin ligase. Cancer Biol Ther 7:1540-3, 2008
53. Moller P, Hagen A I, Apold J, et al: Genetic epidemiology of BRCA mutations—family history detects less than 50% of the mutation carriers. Eur J Cancer 43:1713-7, 2007
54. O'Shaughnessy J, Telli M, Swain S, et al: Phase 3 Study of Iniparib (I) Plus Gemcitabine (G) and Carboplatin (C) in Metastatic Triple-negative Breast Cancer (mTNBC)—Results of an Exploratory Analysis by Prior Therapy European Journal of Cancer 47:S338, 2011
55. Miki Y, Swensen J, Shattuck-Eidens D, Futreal P A, Harshman K, Tavtigian S, Liu Q, Cochran C, Bennett L M, Ding W and et al. (1994). A strong candidate for the breast and ovarian cancer susceptibility gene BRCA1. Science, 266, 66-71.
56. Wooster R, Bignell G, Lancaster J, Swift S, Seal S, Mangion J, Collins N, Gregory S, Gumbs C and Micklem G. (1995). Identification of the breast cancer susceptibility gene BRCA2. Nature, 378, 789-92.
57. Esteller M, Silva J M, Dominguez G, Bonilla F, Matias-Guiu X, Lerma E, Bussaglia E, Prat J, Harkes I C, Repasky E A, Gabrielson E, Schutte M, Baylin S B and Herman J G. (2000). Promoter hypermethylation and BRCA1 inactivation in sporadic breast and ovarian tumors. J Natl Cancer Inst, 92, 564-9.
58. Stephens P J, McBride D J, Lin M L, Varela I, Pleasance E D, Simpson J T, Stebbings L A, Leroy C, Edkins S, Mudie L J, Greenman C D, Jia M, Latimer C, Teague J W, Lau K W, Burton J, Quail M A, Swerdlow H, Churcher C, Natrajan R, Sieuwerts A M, Martens J W, Silver D P, Langerod A, Russnes H E, Foekens J A, Reis-Filho J S, van't Veer L, Richardson A L, Borresen-Dale A L, Campbell P J, Futreal P A and Stratton M R. (2009). Complex landscapes of somatic rearrangement in human breast cancer genomes. Nature, 462, 1005-10.
59. ADDIN ZOTERO_BIBL {“custom”:[ ]} CSL_BIBLIOGRAPHY Birkbak, N. J., Wang, Z. C., Kim, J. Y., Eklund, A. C., Li, Q., Tian, R., Bowman-Colin, C., Li, Y., Greene-Colozzi, A., Iglehart, J. D., et al. (2012). Telomeric allelic imbalance indicates defective DNA repair and sensitivity to DNA-damaging agents. Cancer Discovery 2, 366-375.
60. Lee, M.-N., Tseng, R.-C., Hsu, H.-S., Chen, J.-Y., Tzao, C., Ho, W. L., and Wang, Y.-C. (2007). Epigenetic inactivation of the chromosomal stability control genes BRCA1, BRCA2, and XRCC5 in non-small cell lung cancer. Clin. Cancer Res. 13, 832-838.

1. A method for preparing a DNA fraction for classifying a tumor of a patient, the method comprising:
(a) extracting DNA from a sample from the tumor from the patient;
(b) contacting the extracted DNA with at least 500 oligonucleotides capable of hybridizing to a plurality of polymorphic regions of human genomic DNA to detect a number of large scale transitions (LSTs) per genome; and
(c) analyzing the number of LSTs detected in step (b), and classifying the tumor as responsive to a PARP inhibitor and/or an alkylating agent when the number of LSTs is greater than a predetermined threshold number of LSTs,
wherein an LST is a breakpoint between two genomic regions of different copy number, each such genomic region greater than or equal to 3 and less than 6 megabases long.

2. The method of claim 1, wherein said PARP inhibitor and/or alkylating agent is selected from the group consisting of iniparib, olaparib, rucaparib, CEP 9722, MK 4827, BMN-673, 3-aminobenzamide, platinum complexes, chlormethine, chlorambucil, melphalan, cyclophosphamide, ifosfamide, estramustine, carmustine, lomustine, fotemustine, streptozocin, busulfan, pipobroman, procarbazine, dacarbazine, thiotepa and temozolomide.

3. The method of claim 1, wherein the cancer is selected from breast cancer, ovary cancer, pancreas cancer, head and neck carcinoma and melanoma.

4. The method of claim 1, wherein the cancer is breast cancer.

5. The method of claim 1, wherein the cancer is basal-like breast cancer.

6. The method of claim 1, wherein the patient is identified by detecting, in the tumor sample, the number of LSTs per genome.

7. The method of claim 6, wherein the number of LSTs per genome is detected by detecting copy number and loss of heterozygosity (LOH) status for at least 500 Single Nucleotide Polymorphism (SNP) loci.

8. The method of claim 6, wherein the number of LSTs per genome is detected by detecting copy number and loss of heterozygosity (LOH) status for at least 3,000 Single Nucleotide Polymorphism (SNP) loci.

9. The method of claim 6, wherein the number of LSTs per genome is detected by comparative genomic hybridization (CGH) array, Single Nucleotide Polymorphism (SNP) array, or sequencing of polymorphic loci.
